(19) United States
(12) Patent Application Publication
(10) Pub. No.: US 2009/0036679 A1
(43) Pub. Date: Feb. 5, 2009

(54) METHOD FOR RESOLVING ENANTIOMERS FROM RACEMIC MIXTURE HAVING CHIRAL CARBON IN ALPHA POSITION OF NITROGEN

(75) Inventors: Soon Kil Ahn, Seoul (KR); Hong Woo Lee, Kyeonggi-do (KR); In Taek Lim, Chungcheongnam-do (KR); Dai Sig Im, Kyeonggi-do (KR); Joong Bok Ahn, Chungcheongnam-do (KR); Jung Hwa Lee, Chungcheongnam-do (KR)

Correspondence Address: LUCAS & MERCANTI, LLP, 475 PARK AVENUE SOUTH, 15TH FLOOR, NEW YORK, NY, 10016, US

(73) Assignee: CHONG KUN DANG PHARMACEUTICAL CORP., Seoul (KR)

(21) Appl. No.: 12/278,073

(22) PCT Filed: Jul. 31, 2006
(86) PCT No.: PCT/KR2006/003006

§ 371 Date: Aug. 1, 2008

(30) Foreign Application Priority Data
Feb. 6, 2006 (KR) 10-2006-0011226

(51) Int. Cl. C07D 411/04; C07D 217/00

(52) U.S. Cl. 544/317; 546/139

(57) ABSTRACT

Disclosed relates to a simplified method for resolving enantiomers by dissolving a racemic mixture having chiral carbon in α-position of nitrogen and an amino acid to prepare a diastereomeric salt, not using catalyses or enzymes, with enhancing the optical purity remarkably. Moreover, the present invention can prepare the enantiomers in large quantities without using expensive catalysts or without controlling the reaction conditions for the activity of enzymes applied.

### TECHNICAL FIELD

The present invention relates to a method for resolving enantiomers from a racemic mixture having chiral carbon in α-position of nitrogen and, more particularly, to a method of resolving enantiomers represented by formula 3 or 4 by forming a diastereomeric salt using a racemic mixture, represented by formula 1 or 2 below, having chiral carbon in the α-position of nitrogen, and an amino acid having optical activity.

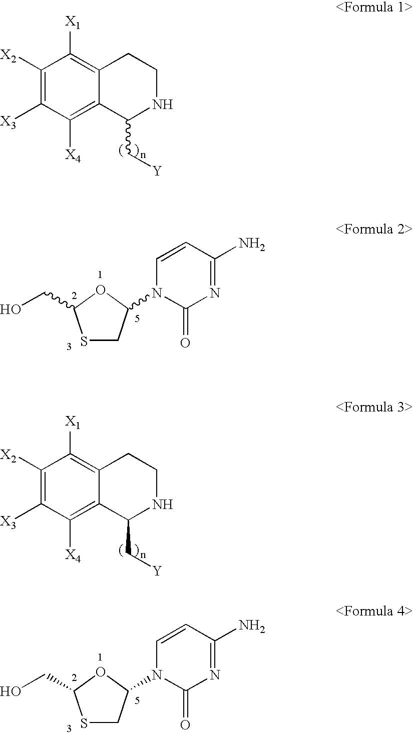

wherein X₁, X₂, X₃ and X₄ are independently selected from the group consisting of hydrogen, halogen, C₁-C₄ alkyl group, hydroxy group and C₁-C₄ alkoxy group; Y represents a phenyl group substituted by at least one substituent selected from the group consisting of halogen, C₁-C₄ alkyl group, hydroxy group and C₁-C₄ alkoxy group, or a naphthyl group unsubstituted or substituted by at least one substituent selected from the group consisting of halogen, C₁-C₄ alkyl group, hydroxy group and C₁-C₄ alkoxy group; and n denotes an integer of 1 to 3.

### BACKGROUND ART

Enantiomers are two pure compounds that have the same physical properties, such as melting point, boiling point, solubility, density and refractive index, but are completely opposite to each other only in optical rotation. In case of a racemic mixture, the optical rotation becomes zero theoretically, and near zero practically. Due to such difference in optical rotation, the spatial arrangement of substituents of chiral carbon is varied, which results in discrepancy in physiological activity and toxicity between the racemic mixture and the respective enantiomers. For example, even though the enantiomers represented by formula 4 have a uniform efficacy for HIV, (−)-enantiomer having a lower cell toxicity than the other enantiomer is considered as a desirable compound as an antiviral agent. Accordingly, it is important to resolve the enantiomers from the racemic mixture.

Meanwhile, the derivative represented by formula 3 corresponds to an intermediate for the synthesis of (S)-6,7-dihydroxy-1-(α-naphthylmethyl-1,2,3,4-tetrahydroisoquinoline that is a therapeutic agent for the treatment of septicemia. More particularly, α-naphthylacetic acid is condensed with 3,4-dimethoxyphenethylamine to prepare N-(3,4-dimethoxyphenylethyl)(α-naphthyl)acetamide and the resulting compound is then reacted with POC13 to prepare 6,7-dihydroxy-1-(α-naphthylmethyl-1,2,3,4-tetrahydroisoquinoline hydrochloride. Finally, the compound obtained is subjected to a stereoselective reduction using (R,R)-Noyori catalyst, thus synthesizing 6,7-dihydroxy-1-(α-naphthylmethyl-1,2,3,4-tetrahydroisoquinoline. However, since the Noyori catalyst applied thereto can not be provided in mass production and is very expensive accordingly, the compound of formula 3 can not be provided in large quantities.

A method for resolving enantiomers from a racemic mixture using an organic acid, (D)-O,O′-dibenzoyl tartaric acid, has been disclosed (Tetrahedron, 1997, 8(2), 277-281). However, as a result of resolving the compound of formula 3 using such method, the resolving efficiency of the enantiomers is not good.

Moreover, the compound, (2R, cis)-4-amino-1-(2-hydroxymethyl-1,3-oxathiolan-5-yl)(1H)-pyrimidin-2-one represented by formula 4 (hereinafter, referred to as 3TC), has been known as a substance having anti-virus activity for human immunodeficiency virus (HIV), the causative virus of acquired immunodeficiency syndrome (AIDS). The compound of formula 4 can be resolved from the racemic mixture using enzymes. More particularly, the compound of formula 4 can be resolved by dissolving the racemic mixture in a manner publicly known in the art. Especially, the compound of formula 4 can be obtained from the well-known racemic mixture via a chiral HPLC, via a selective catabolism of enantiomers using proper enzymes such as cytidine deaminase or via a selective enzymatic hydrolysis of proper derivatives using 5′-nucleotide (International Publication No. WO/1991/017159, Korean Patent No. 10-0244008). However, such processes of resolving enantiomers using enzymes require a further step of preparing an enzyme solution besides the synthesis of compound and a step of maintaining proper pH and temperature for the activity of enzymes. Moreover, since such resolving methods use enzymes, they can not be carried out in large quantities.

Since the compound represented by formula 3 or 4 above having chiral carbon in the α-position of nitrogen is an intermediate engaged in the synthesis of effective enantiomers or is effective in itself, it is necessary to provide a method of improving the resolution efficiency and optical purity and resolving enantiomers from the racemic mixture represented by formula 1 or 2 in large quantities.

### DISCLOSURE

### [Technical Problem]

To overcome the problems in the related art as described above in detail, the present invention provides a method of resolving pure enantiomers represented by formula 3 or 4 by forming a diastereomeric salt using a racemic mixture, represented by formula 1 or 2, having chiral carbon in the α-position of nitrogen, and an amino acid having optical activity.

### [Technical Solution]

To accomplish the object of the present invention, there is provided a method for resolving enantiomers represented by formula 3 below from a racemic mixture, represented by formula 1 below, having chiral carbon in the α-position of nitrogen. Moreover, the present invention provides a method for resolving enantiomers represented by formula 4 below from a racemic mixture, represented by formula 2 below, having chiral carbon in the α-position of nitrogen.

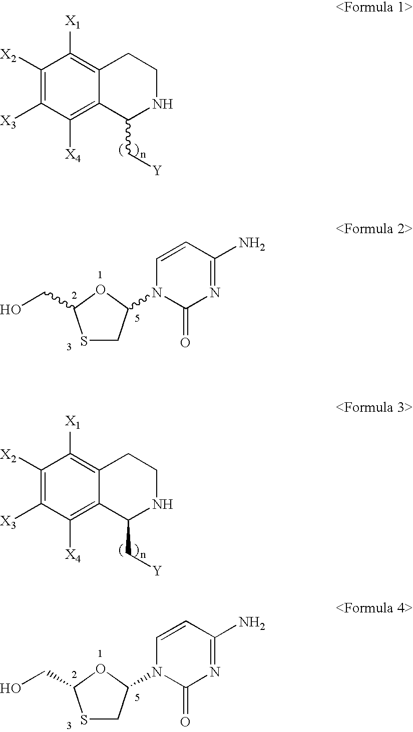

wherein X₁, X₂, X₃ and X₄ are independently selected from the group consisting of hydrogen, halogen, C₁-C₄ alkyl group, hydroxy group and C₁-C₄ alkoxy group; Y represents a phenyl group substituted by at least one substituent selected from the group consisting of halogen, C₁-C₄ alkyl group, hydroxy group and C₁-C₄ alkoxy group, or a naphthyl group unsubstituted or substituted by at least one substituent selected from the group consisting of halogen, C₁-C₄ alkyl group, hydroxy group and C₁-C₄ alkoxy group; and n denotes an integer of 1 to 3.

In formula 1 or 3, more desirably, X₁ and X₄ are hydrogen; X₂, X₃ are methoxy; Y is an unsubstituted naphthyl group or a phenyl group substituted by a methoxy group in the para position.

### [Advantageous Effects]

The present invention provides a simplified method for resolving enantiomers by forming a diastereomeric salt using a racemic mixture and an amino acid, not using catalyses or enzymes, with enhancing the optical purity remarkably. Moreover, the present invention can resolve the enantiomers by forming the diastereomeric salt using the racemic mixture and amino acid having optical activity in large quantities without using expensive catalysts or without controlling the reaction conditions for the activity of enzymes applied.

### [Best Mode]

The method for resolving enantiomers from a racemic mixture having chiral carbon in the α-position of nitrogen in accordance with the present invention comprising the steps of:

dissolving a racemic mixture, represented by formula 1 or 2, having chiral carbon in the α-position of nitrogen, and an amino acid having optical activity in a protic organic solvent (Step 1);

adding an aprotic organic solvent to the reactant solution to crystallize a diastereomeric salt (Step 2); and

obtaining a free amine from the crystallized diastereomeric salt (Step 3).

### [Step 1]

As the protic organic solvent, methanol, ethanol, n-propanol, isopropanol, butanol, ethyleneglycol or their mixture may be used and, more desirably, methanol is used.

Moreover, the amino acid having optical activity may be selected from the group consisting of (R)-N-acetyl-2- phenylglycine, (S)-N-acetyl-2-phenylglycine, (S)-N-acetyltyrosine or (R)-N-acetyltyrosine, (S)-N-acetylphenylalanine or (R)-N-acetylphenylalanine, (S)-N-Boc-2-phenylglycine, (R)-N-Boc-2-phenylglycine, (L)-N-Boc-proline, (D)-N-Boc-proline, (L)-N-Boc-leucine, (D)-N-Boc-leucine, (L)-N-acetyl-valine and (D)-N-acetyl-valine. More desirably, the amino acid having optical activity is selected from the group consisting of (R)-N-acetyl-2-phenylglycine, (S)-N-acetyl-2-phenylglycine, (S)-N-acetyltyrosine, (R)-N-acetyltyrosine, (S)-N-acetylphenylalanine, (R)-N-acetylphenylalanine, (S)-N-Boc-2-phenylglycine and (R)-N-Boc-2-phenylglycine. Most desirably, the amino acid having optical activity is selected from (R)-N-acetyl-2-phenylglycine and (S)-N-acetyl-2-phenylglycine.

The racemic mixture, represented by formula 1 or 2, having chiral carbon in the α-position of nitrogen can be synthesized via well-known methods (Korean Patent Nos. 110506 and 148755, and International Publication No. WO/1991/017159).

The amount of the amino acid having optical activity used in the present invention may be 0.5 to 5.0 for 1.0 equivalent of the racemic mixture of formula 1 or 2 having chiral carbon in the α-position of nitrogen. Desirably, 1.0 of the amino acid is used for 1.0 equivalent of the racemic mixture having chiral carbon in the α-position of nitrogen. If using less than 0.5 equivalents, the enantioselectivity deteriorates, and if using more than 5.0 equivalents, only the preparation cost rises.

### [Step 2]

As the aprotic organic solvent, acetone, methylethylketone, methylisobuthylketone, acetonitrile, ether, ethylacetate, isobuthylacetate, or their mixture may be used and desirably acetone is used.

In the present invention, after dissolving the compound of formula 1 or 2 and an amino acid having optical activity in the protic organic solvent in Step 1, an aprotic organic solvent is added to the reactant solution to crystallize the diasteromeric salt, thus enhancing the optical purity.

Especially, the optical purity obtained by dissolving the compound of formula 1 and the amino acid having optical activity in the protic organic solvent and adding the aprotic organic solvent to the reactant solution to crystallize the diastereomeric salt is remarkably higher than that obtained by crystallizing the diastereomeric salt using a single solvent, not adding the aprotic organic solvent thereto.

Moreover, the optical purity obtained by dissolving the compound of formula 2 and the amino acid having optical activity in the protic organic solvent and adding the aprotic organic solvent to the reactant solution to crystallize the diastereomeric salt is noticeably higher than that obtained by crystallizing the diastereomeric salt using a single solvent, not adding the aprotic organic solvent thereto.

In addition, the aprotic organic solvent is added in the range of 1:1 (v/v) to 1:10 (v/v) to the protic organic solvent added in Step 1 above, desirably, the aprotic organic solvent is added in the range of 1:8 (v/v) to the protic organic solvent and, more desirably, the aprotic organic solvent is added in the range of 1:4 (v/v). If adding the aprotic organic solvent less than 1:1 (v/v) to the protic organic solvent, the reactant solution becomes nearly a saturation state to accelerate the crystallization, thus resulting in the racemic form. Furthermore, if adding the aprotic organic solvent more than 1:10 (v/v) to the protic organic solvent, since the solubility of the racemic mixture decreases due to the increased aprotic organic solvent, the enantiomers are not resolved, thus resulting in the racemic form.

Moreover, the process of crystallizing the diastereomeric salt by adding the aprotic organic solvent to the reactant solution is desirably carried out at −30 to 0° C.

Concretely, when crystallizing the diastereomeric salt at −70 to −30° C., −30 to −20° C., −20 to 0° C. and 0 to 25° C. by dissolving the compound of formula 1 and the amino acid having optical activity in the protic organic solvent and adding the aprotic organic solvent, it can be seen that the optical purity of the diastereomeric salt crystallized at −30 to 0° C. is high.

Furthermore, when crystallizing the diastereomeric salt at −50 to −30° C., −30 to −20° C., −20 to 0° C. and 0 to 25° C. by dissolving the compound of formula 2 and the amino acid having optical activity in the protic organic solvent and adding the aprotic organic solvent, it can be seen that the optical purity of the diastereomeric salt crystallized at −30 to 0° C. is high.

### [Step 3]

The diastereomeric salt can be converted into the respective corresponding free amines in this step. Especially, if the diastereomeric salt obtained in Step 2 above is dissolved in dichloromethane, ether or the same kind of organic solvent and a base is added thereto, the diastereomeric salt is converted into a corresponding free amine.

In case where the present invention is carried out with the racemic mixture of formula 2, the compound of formula 4 having a cis conformation between the substituents of 2-carbon and 5-carbon in the compound of formula 2, with a high optical purity can be obtained.

The method for resolving the compound of formula 3 from the compound of formula 1 provides a higher optical purity than the method for resolving enantiomers using an organic acid, (D)-O,O′-dibenzoyl tartaric acid (Tetrahedron, 1997, 8(2), 277-281). Concretely, as shown in Table 5, the optical purity of Comparative Example 5 is very low compared with the results of Examples 7, 8 and 9. In case of the amino acid having a phenyl group as an organic acid of the present invention, the pure enantiomers are readily resolved.

The optical purity of the free amines should be more than 99% ee. If the optical purity of the derivative is in the range of 98.0% ee to 98.9% ee, Steps 1, 2 and 3 above may be repeated more than one time until the desired purity is obtained.

In accordance with the present invention, a hydrobromide salt may be obtained via a demethylation reaction after resolving enantiomers of tetrahydroisoquinoline derivatives (Korean Patent No. 10-512184). Moreover, it is possible to resolve the optically pure tetrahydroisoquinoline hydrobromide salt freely to be converted into methanesulfonate.

The method of the present invention will now be described as the following non-limited example and is carried out according to the above steps using the derivatives represented by formula 1 (Korean Patent No. 148755) or formula 2 (International Publication No. WO/1991/17159 or Korean Patent No. 244008) as a starting material.

### [Mode for Invention]

Hereinafter, the present invention will now be described more fully with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

### EXAMPLE 1

### (S)-6,7-dimethoxy-1-(α-naphthylmethyl)-1,2,3,4-tetrahydroisoquinoline

662 g (1.99 mol) of racemic mixture, 6,7-dimethoxy-1-(α-naphthylmethyl)-1,2,3,4-tetrahydroisoquinoline was dissolved in 2.38 L of methanol at a 20 L Round-bottom flask. Subsequently, 384 g (1.99 mol) of (R)-N-acetyl-2-phenylglycine was added thereto and dissolved. 9.54 L of acetone was added to the reactant solution and left as it was at −30 to −20° C. for 48 hours to generate solids (crystallized diasteromeric salts). The generated solids were filtered and elutriated in 3.96 L of dichloromethane. Then, 1.32 L of 2 N sodium hydroxide solution was added thereto and stirred for 30 minutes. An organic layer was separated and then admixed with magnesium sulfate anhydrous to be dried. The dried organic layer was filtered under reduced pressure and then concentrated under reduced pressure, thus obtaining 198 g of title compound. (yield of 30%).

HPLC purity was measured in the following manner. 20 μl of sample was injected into a column (Kromasil C18, UG 100 Å, 5 μm, 4.6 mmØ×250 mm) and a mixture containing 0.2 M ammonium acetate (pH 4.0) buffer and heptanesulfonate (IPC B7) with methanol (6/4) was used as a mobile phase. The 0.2 M ammonium acetate (pH 4.0) buffer was prepared by putting 15.4 g of ammonium acetate into a 1 L flask to be dissolved with about 900 mL of purified water and adding acetate to calibrate the pH at 4.0, wherein purified water was added to reach the marked line. The temperature of the column and the flow rate were kept at 40° C. and 1.5 mL/min, respectively. The HPLC purity of enantiomers was measured at a wavelength of 284 nm using a UV-spectrophotometer (HPLC purity: 99%).

Optical purity was measured in the following manner. 20 μl of sample was injected into a column (DIACEL CHIRALCEL OD-H, 5 μm, 4.6 mmØ×250 mm) and a solution mixed with n-hexane, isopropanol and diethylamine in the ratio of 40:10:0.05 was used as a mobile phase. The temperature of the column and the flow rate were kept at 25° C. and 0.5 mL/min, respectively. The optical purity of enantiomers was measured at a wavelength of 254 nm using a UV-spectrophotometer (optical purity: 99% ee).

[a]²⁰D=+73.3 (c=0.083, MeOH)

IR (CHCl₃) cm⁻¹: 3420, 2934, 1510, 1222

¹H NMR (400 MHz, CDCl₃) δ: 2.76 (m, 1H), 2.82 (m, 1H), 2.94-2.89 (m, 1H), 3.32-3.23 (m, 2H), 3.75-3.72 (m, 1H), 3.77 (s, 3H), 3.87 (s, 3H), 4.36-4.33 (m, 1H), 6.62 (s, 2H), 7.45-7.37 (m, 2H), 7.57-7.49 (m, 2H), 7.78 (d, 1H, J=8.0 Hz), 7.89 (d, 1H, J=7.8 Hz), 8.18 (d, 1H, J=8.2 Hz)

¹³C NMR (100 MHz, CDCl₃) δ: 29.43, 40.10, 40.63, 55.85, 55.90, 56.03, 109.80, 111.95, 123.77, 125.55, 125.73, 126.10, 127.23, 127.39, 127.99, 128.99, 130.64, 132.19, 134.11, 135.15, 146.99, 147.64

MS m/z (M+H⁺) 334

### EXAMPLES 2, 3 AND 4

Examples 2, 3 and 4 were carried out in the same manner as Example 1, except for using methylethyl ketone in Example 2, Methylisobuthyl ketone in Example 3 and acetonitrile in Example 4 as the aprotic organic solvent, instead of acetone used in Example 1.

### EXAMPLE 5

Example 5 was carried out in the same manner as Example 1, except for the process of crystallization performed at −20 to 0° C., instead of −30 to −20° C. in Example 1.

### EXAMPLE 6

### (R)-6,7-dimethoxy-1-(α-naphthylmethyl)-1,2,3,4-tetrahydroisoquinoline

6.00 g (17.99 mmol) of racemic mixture, 6,7-dimethoxy-1-(α-naphthylmethyl)-1,2,3,4-tetrahydroisoquinoline was dissolved in 20 mL of methanol. Subsequently, 3.48 g (17.99 mmol) of (S)-N-acetyl-2-phenylglycine was added thereto and dissolved. 80 mL of acetone was added to the reactant solution and left as it was at −30 to −20° C. for 48 hours to generate solids (crystallized diasteromeric salts). The generated solids were filtered and elutriated in 20 mL of dichloromethane. Then, 15 mL of 2 N sodium hydroxide solution was added thereto and stirred for 30 minutes. An organic layer was separated and then admixed with magnesium sulfate anhydrous to be dried. The dried organic layer was filtered under reduced pressure and then concentrated under reduced pressure, thus obtaining 1.80 g of title compound (yield of 30%). HPLC purity and optical purity measured in the same manner as Example 1 were more than 99% and 99.4% ee, respectively.

[α]²⁰D=−72.1 (c=0.99, MeOH)

IR (CHCl₃) cm⁻¹: 3420, 2934, 1510, 1222

¹H NMR (400 MHz, CDCl₃) δ: 2.76 (m, 1H), 2.82 (m, 1H), 2.94-2.89 (m, 1H), 3.32-3.23 (m, 2H), 3.75-3.72 (m, 1H), 3.77 (s, 3H), 3.87 (s, 3H), 4.36-4.33 (m, 1H), 6.62 (s, 2H), 7.45-7.37 (m, 2H), 7.57-7.49 (m, 2H), 7.78 (d, 1H, J=8.0 Hz), 7.89 (d, 1H, J=7.8 Hz), 8.18 (d, 1H, J=8.2 Hz)

¹³C NMR (100 MHz, CDCl₃) δ: 29.43, 40.10, 40.63, 55.85, 55.90, 56.03, 109.80, 111.95, 123.77, 125.55, 125.73, 126.10, 127.23, 127.39, 127.99, 128.99, 130.64, 132.19, 134.11, 135.15, 146.99, 147.64

MS m/z (M+H⁺) 334

### EXAMPLES 7, 8 AND 9

Examples 7, 8 and 9 were carried out in the same manner as Example 1, except for using (R)-N-acetyltyrosine in Example 7, (R)-N-acetylphenylalanine in Example 8 and (R)-N-Boc-2-phenylglycine in Example 9, instead of (R)-N-acetyl-2-phenylglycine used in Example 1.

### EXAMPLE 10

### (S)-6,7-dimethoxy-1-(para-methoxyphenylmethyl)-1,2,3,4-tetrahydroisoquinoline

5.00 g (15.95 mmol) of racemic mixture, 6,7-dimethoxy-1-(α-naphthylmethyl)-1,2,3,4-tetrahydroisoquinoline was dissolved in 16 mL of methanol. Subsequently, 3.08 g (15.95 mmol) of (R)-N-acetyl-2-phenylglycine was added thereto and dissolved. 64 mL of acetone was added to the reactant solution and left as it was at −30 to −20° C. for 24 hours to generate solids (crystallized diasteromeric salts). The generated solids were filtered and elutriated in 20 mL of dichloromethane. Then, 15 mL of 2 N sodium hydroxide solution was added thereto and stirred for 30 minutes. An organic layer was separated and then admixed with of magnesium sulfate anhydrous to be dried The dried organic layer was filtered under reduced pressure and then concentrated under reduced pressure, thus obtaining 1.50 g of title compound (yield of 30%). HPLC purity and optical purity measured in the same manner as Example 1 were 99% and 98% ee, respectively. As a result of repeating the above process, 1.3 g of target title compound having an optical purity 99.6% ee was obtained.

[α]²⁸D=+25.6 (c=0.052, MeOH)

IR (CHCl₃) cm⁻¹: 3421, 2930, 1512, 1223

¹H NMR (400 MHz, CDCl₃) δ: 3.0-3.5(m, 5H), 3.61(s, 3H), 3.85(m, 6H), 4.72(s, 1H), 6.60 (s, 1H), 6.72 (s, 1H), 6.88 (s, 1H), 7.04 (m, 2H), 7.40 (m, 2H),

MS m/z (M+H⁺) 314

### EXAMPLE 11

### (R)-6,7-dimethoxy-1-(para-methoxyphenylmethyl)-1,2,3,4-tetrahydroisoquinoline

Example 11 was carried out in the same manner as Example 10, except for using 3.08 g (15.95 mmol) of (S)-N-acetyl-2-phenylglycine, instead of 3.08 g (15.95 mmol) of (R)-N-acetyl-2-phenylglycine used in Example 10. As a result, 1.60 g of title compound was obtained (yield: 32%). HPLC purity and optical purity measured in the same manner as Example 1 were 99% and 99% ee, respectively.

[a]²⁸D=−25.0 (c=0.05, MeOH)

IR (CHCl₃) cm⁻¹: 3421, 2930, 1512, 1223

¹H NMR (400 MHz, CDCl₃) δ: 3.0-3.5 (m, 5H), 3.61 (s, 3H), 3.85 (m, 6H), 4.72 (s, 1H), 6.60 (s, 1H), 6.72 (s, 1H), 6.88 (s, 1H), 7.04 (m, 2H), 7.40 (m, 2H),

MS m/z (M+H⁺) 314

### EXAMPLE 12

### (S)-6,7-dimethoxy-1-(β-naphthylmethyl)-1,2,3,4-tetrahydroisoquinoline

6.00 g (17.99 mmol) of racemic mixture, 6,7-dimethoxy-1-(β-naphthylmethyl)-1,2,3,4-tetrahydroisoquinoline was dissolved in 20 mL of methanol. Subsequently, 3.48 g (17.99 mol) of (R)-N-acetyl-2-phenylglycine was added thereto and dissolved. 80 mL of acetone was added to the reactant solution and left as it was at −30 to −20° C. for 24 hours to generate solids (crystallized diasteromeric salts). The generated solids were filtered and elutriated in 3.96 L of dichloromethane. Then, 1.32 L of 2 N sodium hydroxide solution was added thereto and stirred for 30 minutes. An organic layer was separated and then admixed with magnesium sulfate anhydrous to be dried. The dried organic layer was filtered under reduced pressure and then concentrated under reduced pressure, thus obtaining 1.80 g of title compound (yield of 30%). HPLC purity and optical purity measured in the same manner as Example 1 were more than 99% and 99.6% ee, respectively.

[α]²⁹D=+33.0 (c=0.052, CDCl₃)

IR (CHCl₃) cm⁻¹: 3421, 2934, 1511, 1221

¹H NMR (400 MHz, CDCl₃) δ: 3.13 (m, 1H), 3.27 (m, 1H), 3.44 (m, 1H), 3.57 (m, 1H), 3.69 (m, 1H), 3.78 (m, 1H), 3.78 (s, 3H), 3.87 (s, 3H), 4.32 (m, 1H), 4.89 (m, 1H), 5.30 (s, 1H), 6.56 (s, 1H), 7.16 (m, 1H), 7.34 (m, 1H), 7.45 (m, 2H), 7.78 (m, 1H), 7.85 (m, 1H), 8.25 (m, 1H)

MS m/z (M+H⁺) 334

### EXAMPLE 13

### (R)-6,7-dimethoxy-1-(β-naphthylmethyl)-1,2,3,4-tetrahydroisoquinoline

Example 13 was performed in the same manner as Example 12, except for using 3.48 g of 17.99 mmol (S)-N-acetyl-2-phenylglycine, instead of 3.48 g (17.99 mmol) of (R)-N-acetyl-2-phenylglycine used in Example 12. As a result, 1.80 g of title compound was obtained (yield: 30%). HPLC purity and optical purity measured in the same manner as Example 1 were 99% and 99.8% ee, respectively.

[a]²⁹D=−33.1 (c=0.049, CDCl₃)

IR (CHCl₃) cm⁻¹: 3421, 2934, 1511, 1221

¹H NMR (400 MHz, CDCl₃) δ: 3.13 (m, 1H), 3.27 (m, 1H), 3.44 (m, 1H), 3.57 (m, 1H), 3,69 (m, 1H), 3.78 (m, 1H), 3.78 (s, 3H), 3.87 (s, 3H), 4.32 (m, 1H), 4.89 (m, 1H), 5.30 (s, 1H), 6.56 (s, 1H), 7.16 (m, 1H), 7.34 (m, 1H), 7.45 (m, 2H), 7.78 (m, 1H), 7.85 (m, 1H), 8.25 (m, 1H)

MS m/z (M+H⁺) 334

### EXAMPLE 14

### (S)-6,7-dihydroxy-1-(α-naphthylmethyl)-1,2,3,4-tetrahydroisoquinoline hydrobromide salt

198 g (0.59 mol) of (S)-6,7-dimethoxy-1-(α-naphthylmethyl)-1,2,3,4-tetrahydroisoquinoline was put into a 20 L round-bottom flask and melted in 8 L of chloroform. Then, 464 g (1.48 mol) of borontribromide methylsulfide was slowly added thereto. The reactant solution was stirred to reflux for 36 hours and dried at room temperature. Subsequently, 4.80 L of methanol was slowly added thereto and stirred for one hour. The solvent was concentrated under reduced pressure and removed. Solids obtained were elutriated in 1.20 L of isopropylacetate and vacuum dried at room temperature, thus obtaining 195 g of title compound (yield: 85%). HPLC purity measured in the same manner as Example 1 was more than 99%.

IR (KBr) cm⁻¹: 3423, 1618, 1195, 1045

¹H NMR (400 MHz, DMSO-d₆) δ: 2.22 (s, 3H), 2.77-2.73 (m, 1H), 2.90-2.85 (m, 1H), 3.16-3.16 (m, 1H), 3.43-3.35 (m, 2H), 3.76-3.71 (m, 1H), 4.60 (t, 1H), 6.34 (s, 1H), 6.53, (s, 1H), 7.32 (d, 1H), 7.42 (t, 1H), 7.58-7.50 (m, 2H), 7.86 (d, 1H, J=8.0 Hz), 7.94 (d, 1H, J=7.8 Hz), 8.11 (d, 1H, J=8.2 Hz), 8.76 (s, 1H), 9.07 (s, 1H), 9.08 (s, 1H)

¹³C NMR (100 MHz, DMSO-d₆) δ: 24.65, 25.91, 37.25, 54.65, 62.44, 114.11, 115.70, 122.68, 122.93, 123.99, 126.06, 126.34, 126.93, 128.46, 129.03, 129.31, 131.98, 132.09, 134.14, 144.27, 144.40, 145.54, 145.68

MS m/z (M+H⁺) 306

### EXAMPLE 15

### (S)-6,7-dihydroxy-1-(α-naphthylmethyl)-1,2,3,4-tetrahydroisoquinoline methanesulfonate

195 g (0.50 mol) of (S)-6,7-dihydroxy-1-(α-naphthylmethyl)-1,2,3,4-tetrahydroisoquinoline hydrobromide salt was dissolved in 8 L of dichloromethane/methanol (3/1, v/v). Then, 5 L of 5% sodium bicarbonate was added thereto and stirred for 20 minutes to separate an organic layer. Here, the pH of watery layer was set at about 8 and the water layer was reextracted thrice by 2 L. 6 L of saturated sodium bicarbonate solution was added to the collected organic layers and stirred for 20 minutes to separate an organic layer again. After separating the organic layer, magnesium sulfate anhydrous was added thereto and dried. Subsequently, the dried organic layer was filtered under reduced pressure and the solvent was concentrated under reduced pressure and removed. The concentrated solids were melted in 0.50 L of dichloromethane/methanol (¼) and then the dichloromethane solvent was concentrated under reduced pressure and removed. Subsequently, 114 g (1.19 mol) of methanesulfonate was added thereto and stirred. When solids were precipitated, 1.60 L of diethylether was added thereto to precipitate all solids and then stirred for 12 hours. Subsequently, the reactant solution was stirred at 0° C. for two hours and filtered under reduced pressure. Solids obtained were dried under reduced pressure at room temperature and then dried in a reduced pressure oven dryer at 70° C. for four days, thus obtaining 200 g of title compound (yield: 99%). HPLC purity measured in the same manner as Example 1 was more than 99%. Optical purity was measured in the following manner. 20 μl of sample was injected into a column (Phenomenex Chirex (S)-LEU and (R)-LEU (UG 100 Å, 5 μm, 4.6 mmØ×250 mm) and a mixed solution (n-hexane:ethanol=4:1) containing IPC B7 was used as a mobile phase. The temperature of the column and the flow rate were kept at 25° C. and 0.9 mL/min, respectively. The optical purity of enantiomers was measured at a wavelength of 254 nm using a UV-spectrophotometer (optical purity: 100% ee).

[a]²⁰D=+74.3 (c=0.25, CH₃CN)

IR (KBr, cm⁻¹): 3423, 1618, 1195, 1045

UV nm: 225 nm, 284 nm

¹H NMR (400 MHz, DMSO-d₆) δ: 2.22 (s, 3H), 2.77-2.73 (m, 1H), 2.90-2.85 (m, 1H), 3.16-3.16 (m, 1H), 3.43-3.35 (m, 2H), 3.76-3.71 (m, 1H), 4.60 (t, 1H), 6.34 (s, 1H), 6.53, (s, 1H), 7.32 (d, 1H), 7.42 (t, 1H), 7.58-7.50 (m, 2H), 7.86 (d, 1H, J=8.0 Hz), 7.94 (d, 1H, J=7.8 Hz), 8.11 (d, 1H, J=8.2 Hz), 8.76 (s, 1H), 9.07 (s, 1H), 9.08 (s, 1H)

¹³C NMR (100 MHz, DMSO-d₆) δ: 24.65, 25.91, 37.25, 54.64, 62.11, 114.11, 115.70, 122.68, 122.93, 123.99, 126.06, 126.34, 126.93, 128.46, 129.03, 129.31, 131.98, 132.09, 134.14, 144.27, 144.40, 145.54, 145.68

HR-MS Calcd. for C₂₀H₂₀NO₂: MH+, 306.1494. Found: 306.1490 (MH⁺)

Calculated values of elementary analysis for C₂₁H₂₃NO₅S: C, 62.0; H, 6.0; N, 3.4; S, 7.9. (Measured values: C, 61.6; H, 5.8; N, 3.5; S, 7.8)

### EXAMPLE 16

### (2R)-cis-4-amino-1-(2-hydroxymethyl-1,3-oxathiolane-5-il)-(1H)-pyrimidin-2-one

5.0 g (21.8 mmol) of racemic mixture, cis-4-amino-1-(2-hydroxymethyl-1,3-oxathiolane-5il)-(1H)-pyrimidin-2-one was dissolved in 20 mL of methanol. Subsequently, 4.21 g (21.8 mmol) of (S)-N-acetyl-2-phenylglycine was added thereto and dissolved. 80 mL of acetone was added to the reactant solution and left as it was at −30 to −20° C. for 48 hours to generate solids (crystallized diasteromeric salts). The generated solids were filtered and elutriated in 3.96 L of dichloromethane. Then, 1.32 L of 2 N sodium hydroxide solution was added thereto and stirred for 30 minutes. An organic layer was separated and then admixed with magnesium sulfate anhydrous to be dried. The dried organic layer was filtered under reduced pressure and then concentrated under reduced pressure, thus obtaining 1.60 g of title compound (yield of 32%). HPLC purity was measured in the following manner. 20 μl of sample was injected into a column (Spherisorb ODS-2 (5 μm, 4.6 mmØ×150 mm) and ammonium dihydrogen phosphate+5% acetonitrile was used as a mobile phase. The temperature of the column and the flow rate were kept at 25° C. and 1.5 ml/min, respectively. The HPLC purity of enantiomers was measured at a wavelength of 270 nm using a UV-spectrophotometer (HPLC purity: more than 99%).

Optical purity was measured in the following manner. 20 μl of sample was injected into a column (Cyclobond I Acetyl (4.6 mmØ×250 mm) and 0.2% triethylammonium acetate (pH 7.2) was used as a mobile phase. The temperature of the column and the flow rate were kept at 25° C. and 1.0 mL/min, respectively. The optical purity of enantiomers was measured at a wavelength of 270 nm using a UV-spectrophotometer (optical purity: 99.8% ee).

[α]²⁰D=+138 (c 0.98, MeOH)

IR (KBr) cm⁻¹: 3340, 1665, 1480

¹H NMR (400 MHz, DMSO-d₆) δ: 3.05 (m, 1H), 3.41 (m, 1H), 3.73 (m, 2H), 5.18 (m, 1H), 5.29 (m, 1H), 5.73 (m, 1H), 6.22 (m, 1H), 7.20 (br s, 2H), 7.80 (m, 1H)

MS m/z (M+H⁺) 230

### EXAMPLES 17, 18 AND 19

Examples 17, 18 and 19 were carried out in the same manner as Example 16, except for using (S)-N-acetyltyrosine in Example 17, (S)-N-acetylphenylalanine in Example 18 and (S)-N-Boc-2-phenylglycine in Example 19, instead of (S)-N-acetyl-2-phenylglycine used in Example 16. Optical purity was measured in the same manner as Example 16 above.

### EXAMPLE 20

Example 20 was carried out in the same manner as Example 16, except for the process of crystallization performed at −20 to 0° C., instead of −30 to −20° C. in Example 16.

### EXAMPLE 21

### (2S)-cis-4-amino-1-(2-hydroxymethyl-1,3-oxathiolane-5-il)-(1H)-pyrimidin-2-one

5.50 g (23.99 mmol) of racemic mixture, cis-4-amino-1-(2-hydroxymethyl-1,3-oxathiolane-5-il)-(1H)-pyrimidin-2-one was dissolved in 20 mL of methanol. Subsequently, 4.63 g (23.99 mmol) of (R)-N-acetyl-2-phenylglycine was added thereto and dissolved. 80 mL of acetone was added to the reactant solution and left as it was at −30 to −20° C. for 48 hours to generate solids (crystallized diasteromeric salts). The generated solids were filtered and elutriated in 3.96 L of dichloromethane. Then, 1.32 L of 2 N sodium hydroxide solution was added thereto and stirred for 30 minutes. An organic layer was separated and then admixed with magnesium sulfate anhydrous to be dried. The dried organic layer was filtered under reduced pressure and then concentrated under reduced pressure, thus obtaining 1.40 g of title compound (yield of 25%). HPLC purity and optical purity measured in the same manner as Example 16 were more than 99% and 99.2% ee, respectively.

[α]²⁰D=−135 (c=0.86, MeOH)

IR (KBr) cm⁻¹: 3340, 1665, 1480

¹H NMR (400 MHz, DMSO-d₆) δ: 3.05 (m, 1H), 3.41 (m, 1H), 3.73 (m, 2H), 5.18 (m, 1H), 5.29 (m, 1H), 5.73 (m, 1H), 6.22 (m, 1H), 7.20 (br s, 2H), 7.80 (m, 1H)

MS m/z (M+H⁺) 230

### COMPARATIVE EXAMPLE 1

Comparative Example 1 was performed in the same manner as Example 1 above, except for not using the aprotic organic solvent, acetone.

### COMPARATIVE EXAMPLE 2

Comparative Example 2 was performed in the same manner as Example 1, except for using ethanol, instead of the protic organic solvent, methanol, and except for not using the aprotic organic solvent.

### COMPARATIVE EXAMPLE 3

Comparative Example 3 was carried out in the same manner as Example 1, except for the process of crystallization performed at 0 to 25° C., instead of −30 to −20° C. in Example 1.

### COMPARATIVE EXAMPLE 4

Comparative Example 4 was carried out in the same manner as Example 1, except for the process of crystallization performed at −70 to −30° C., instead of −30 to −20° C. in Example 1.

### COMPARATIVE EXAMPLE 5

Comparative Example 5 was performed in the same manner as Example 1, except for using (D)-O,O′-dibenzoyl tartaric acid, instead of (R)-N-acetyl-2-phenylglycine used in Example 1.

### COMPARATIVE EXAMPLE 6

Comparative Example 6 was performed in the same manner as Example 16, except for not using the aprotic organic solvent, acetone.

### COMPARATIVE EXAMPLE 7

Comparative Example 7 was performed in the same manner as Example 16, except for using ethanol, instead of the protic organic solvent, methanol, and except for not using the aprotic organic solvent.

### COMPARATIVE EXAMPLE 8

Comparative Example 8 was performed in the same manner as Example 16, except for using (L)-O,O′-dibenzoyl tartaric acid, instead of (S)-N-acetyl-2-phenylglycine used in Example 16.

### COMPARATIVE EXAMPLE 9

Comparative Example 9 was carried out in the same manner as Example 16 except for the process of crystallization performed at 0 to 25° C., instead of −30 to −20° C. in Example 1.

### COMPARATIVE EXAMPLE 10

Comparative Example 10 was carried out in the same manner as Example 16, except for the process of crystallization performed at −50 to −30° C., instead of to −20° C. in Example 1.

### EXPERIMENTAL EXAMPLE 1

### Effects of Aprotic Organic Solvent on the Process of Resolving Enantiomers

An experiment was performed in order to examine the effects of the usages of the aprotic organic solvent on the optical purities in the process Examples 1, 2, 3 and 4 and Comparative Examples 1 and 2 were all carried out under the same conditions, except for the protic organic solvent and the aprotic organic solvent applied thereto. The respective optical purities obtained were depicted in Table 1 below.

TABLE 1

| | | Ratio of | | | |
|---|---|---|---|---|---|
| | Organic | organic | Temperature | Ratio of | Optical |
| Example | Solvent | solvent (V/V). | (° C.) | (R)/(S) | purity (% ee) |
| Example 1 | Methanol/Acetone | 1/4 | −30 to −20 | 0.1/99.9 | 99.8 |
| Example 2 | Methanol/Methylethyl | 1/4 | −30 to −20 | 1/99 | 98 |
| | ketone | | | | |
| Example 3 | Methanol/Methyliso- | 1/4 | −30 to −20 | 4/96 | 92 |
| | buthylKetone | | | | |
| Example 4 | Methanol/Acetonitrile | 1/4 | −30 to −20 | 5/95 | 90 |
| Comparative | Methanol | — | −30 to −20 | 10/90 | 80 |
| Example 1 | | | | | |
| Comparative | Ethanol | — | −30 to −20 | 20/80 | 60 |
| Example 2 | | | | | |

As shown in Table 1, it could be seen that the optical purities obtained by using the aprotic organic solvent in Examples 1, 2, 3 and 4 were very higher than those obtained without using the aprotic organic solvent in Comparative Examples 1 and 2 during the process of crystallizing the diastereomeric salts.

Moreover, an experiment was performed in order to examine the effects of the usages of the aprotic organic solvent on the optical purities in the process of resolving the compound of formula 4 from the racemic mixture of formula 2. Examples 16 and Comparative Examples 6 and 7 were all carried out under the same conditions, except for the protic organic solvent and the aprotic organic solvent applied thereto. The respective optical purities obtained were depicted in Table below.

TABLE 2

| | | Ratio of | | | |
|---|---|---|---|---|---|
| | Organic | organic | Temperature | Ratio of | Optical |
| Example | Solvent | solvent (V/V). | (° C.) | (R)/(S) | purity (% ee) |
| Example 16 | Methanol/Acetone | 1/4 | −30 to −20 | 0.1/99.9 | 99.8 |
| Comparative | Methanol | — | −30 to −20 | 10/90 | 80 |
| Example 6 | | | | | |
| Comparative | Ethanol | — | −30 to −20 | 30/70 | 40 |
| Example 7 | | | | | |

As shown in Table 2, it could be learned that the optical purity obtained by using the aprotic organic solvent in Example 16 was very higher than those obtained without using the aprotic organic solvent in Comparative Examples 6 and during the process of crystallizing the diastereomeric salts.

### EXPERIMENTAL EXAMPLE 2

### Effects of Crystallizing Temperatures on the Process of Resolving Enantiomers

An experiment was performed in order to examine the effects of the crystallizing temperatures on the optical purities in the process of resolving the compound of formula 3 from the racemic mixture of formula 1. Examples 5 and 6 and Comparative Examples 3 and 4 were all carried out under the same conditions, except for the varied temperatures for crystallizing the diastereomeric salts. The respective optical purities obtained were depicted in Table 3 below.

TABLE 3

| | | Ratio of | | | |
|---|---|---|---|---|---|
| | Organic | organic | Temperature | Ratio of | Optical |
| Example | Solvent | solvent (V/V). | (° C.) | (R)/(S) | purity (% ee) |
| Example 5 | Methanol/Acetone | 1/4 | −20 to 0 | 99.5/0.5 | 99 |
| Example 6 | Methanol/Acetone | 1/4 | −30 to −20 | 99.7/0.3 | 99.4 |
| Comparative | Methanol/Acetone | 1/4 | 0 to 25 | 90/10 | 80 |
| Example 3 | | | | | |
| Comparative | Methanol/Acetone | 1/4 | −70 to −30 | 80/20 | 60 |
| Example 4 | | | | | |

As shown in Table 3, it could be understood that the optical purities obtained at −30 to 0° C. in Examples 5 and 6 were very higher than those obtained at −70 to −30° C. and at 0 to 25° C. in Comparative Examples 3 and 4 during the process of crystallizing the diastereomeric salts.

In addition, experiment was performed in order to examine the effects of the crystallizing temperatures on the optical purities in the process of resolving the compound of formula 4 from the racemic mixture of formula 2. Examples 16 and 20 and Comparative Examples 9 and 10 were all carried out under the same conditions, except for the varied temperatures for crystallizing the diastereomeric salts. The respective optical purities obtained were depicted in Table 4 below.

TABLE 4

| | | Ratio of | | | |
|---|---|---|---|---|---|
| | Organic | organic | Temperature | Ratio of | Optical |
| Example | Solvent | solvent (V/V). | (° C.) | (R)/(S) | purity (% ee) |
| Example 16 | Methanol/Acetone | 1/4 | −30 to −20 | 0.1/99.9 | 99.8 |
| Example 20 | Methanol/Acetone | 1/4 | −20 to 0 | 0.5/99.5 | 99 |
| Comparative | Methanol/Acetone | 1/4 | 0 to 25 | 45/55 | 10 |
| Example 9 | | | | | |
| Comparative | Methanol/Acetone | 1/4 | −50 to −30 | 30/70 | 40 |
| Example 10 | | | | | |

As shown in Table 4, it could be aware that the optical purities obtained at −30 to 0° C. in Examples 16 and 20 were very higher than those obtained at −50 to −30° C. and at 0 to 25° C. in Comparative Examples 9 and 10 during the process of crystallizing the diastereomeric salts.

### EXPERIMENTAL EXAMPLE 3

### Effects of Amino Acid Having Optical Activity on the Process of Resolving Enantiomers

An experiment was performed in order to examine the effects of the amino acid having optical activity on the optical purities in the process of resolving the compound of formula 3 from the racemic mixture of formula 1. Examples 7, 8 and 9 and Comparative Example 5 were all carried out under the same conditions, except for the organic acid (amino acid) used therein. The respective optical purities obtained were depicted in Table 5 below.

TABLE 5

| | | Ratio of | Optical |
|---|---|---|---|
| Example | Amino acid(organic acid) | (R)/(S) | purity (% ee) |
| Example 5 | (R)—N-acetyltyrosine | 0.3/99.7 | 99.4 |
| Example 6 | (R)—N-acetylphenylalanine | 0.5/99.5 | 99 |
| Comparative | (R)—N-Boc-2-phenylglycine | 0.5/99.5 | 99 |
| Example 3 | | | |
| Comparative | (D)-O,O′-dibenzoyl | 45/55 | 10 |
| Example 4 | tartaric acid | | |

As shown in Table 5, it could be found that the optical purities obtained by using the amino acid having optical activity in Examples 7, 8 and 9 in accordance with the present invention were very higher than that obtained by using the conventional (D)-OO′-dibenzoyl tartaric acid in Comparative Examples 5.

Furthermore, an experiment was performed in order to examine the effects of the amino acid having optical activity on the optical purities in the process of resolving the compound of formula 4 from the racemic mixture of formula 2. Examples 17, 18 and 19 and Comparative Example 8 were all carried out under the same conditions, except for the organic acid (amino acid) used therein. The respective optical purities obtained were depicted in Table 6 below.

TABLE 6

| | | | |
|---|---|---|---|
| | | Ratio of | Optical |
| Example | Amino acid(organic acid) | (R)/(S) | purity (% ee) |

| | | | |
|---|---|---|---|
| Example 17 | (S)—N-acetyltyrosine | 99.5/0.5 | 99 |
| Example 18 | (S)—N-acetylphenylalanine | 99.5/0.5 | 99 |
| Example 19 | (S)—N-Boc-2-phenylglycine | 99.6/0.4 | 99.2 |
| Comparative | (L)-O,O′-dibenzoyl | 60/40 | 20 |
| Example 8 | tartaric acid | | |

As shown in Table 6, it could be confirmed that the optical purities obtained by using the amino acid having optical activity in Examples 17, 18 and 19 in accordance with the present invention were very higher than that obtained by using the conventional (L)-O,O′-dibenzoyl tartaric acid in Comparative Examples 8.

1. A method for resolving enantiomers from a racemic mixture having chiral carbon in α-position of nitrogen comprising the steps of:
dissolving a racemic mixture, represented by formula 1 or 2, having chiral carbon in the α-position of nitrogen, and an amino acid having optical activity in a protic organic solvent (Step 1);
adding an aprotic organic solvent to the reactant solution to crystallize a diastereomeric salt (Step 2); and
obtaining a free amine from the crystallized diastereomeric salt (Step 3). 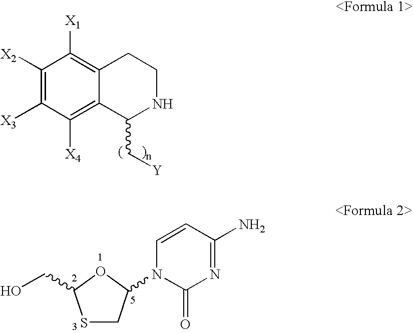
wherein X₁, X₂, X₃ and X₄ are independently selected from the group consisting of hydrogen, halogen, C₁-C₄ alkyl group, hydroxy group and C₁-C₄ alkoxy group; Y represents a phenyl group substituted by at least one substituent selected from the group consisting of halogen, C₁-C₄ alkyl group, hydroxy group and C₁-C₄ alkoxy group, or a naphthyl group unsubstituted or substituted by at least one substituent selected from the group consisting of halogen, C₁-C₄ alkyl group, hydroxy group and C₁-C₄ alkoxy group; and n denotes an integer of 1 to 3.

2. The method for resolving enantiomers from a racemic mixture having chiral carbon in α-position of nitrogen as recited in claim 1, wherein X1 and X4 are hydrogen; X2 and X3 are methoxy; Y is a naphthyl group unsubstituted or a phenyl group substituted by a methoxy group in para position; and n is an integer of 1.

3. The method for resolving enantiomers from a racemic mixture having chiral carbon in α-position of nitrogen as recited in claim 1, wherein the protic organic solvent is selected from the group consisting of methanol, ethanol, n-propanol, isopropanol, butanol, ethyleneglycol and their mixture.

4. The method for resolving enantiomers from a racemic mixture having chiral carbon in α-position of nitrogen as recited in claim 1,
wherein the amino acid having optical activity is selected from the groups consisting of (R)-N-acetyl-2-phenylglycine, (S)-N-acetyl-2-phenylglycine, (S)-N-acetyltyrosine or (R)-N-acetyltyrosine, (S)-N-acetylphenylalanine or (R)-N-acetylphenylalanine, (S)-N-Boc-2-phenylglycine, (R)-N-Boc-2-phenylglycine, (L)-N-Boc-proline, (D)-N-Boc-proline, (L)-N-Boc-leucine, (D)-N-Boc-leucine, (L)-N-acetyl-valine and (D)-N-acetyl-valine, and more desirably, the amino acid having optical activity is selected from the group consisting of (R)-N-acetyl-2-phenylglycine, (S)-N-acetyl-2-phenylglycine, (S)-N-acetyltyrosine, (R)-N-acetyltyrosine, (S)-N-acetylphenylalanine, (R)-N-acetylphenylalanine, (S)-N-Boc-2-phenylglycine and (R)-N-Boc-2-phenylglycine.

5. The method for resolving enantiomers from a racemic mixture having chiral carbon in α-position of nitrogen as recited in claim 1,
wherein the aprotic organic solvent is selected from the group consisting of acetone, methylethylketone, methylisobuthylketone, acetonitrile, ether, ethylacetate, isobuthylacetate and their mixture.

6. The method for resolving enantiomers from a racemic mixture having chiral carbon in α-position of nitrogen as recited in claim 1,
wherein the aprotic organic solvent is added in the range of 1:1 (v/v) to 1:10 (v/v) to the protic organic solvent.

7. The method for resolving enantiomers from a racemic mixture having chiral carbon in α-position of nitrogen as recited in claim 1,
wherein, in Step 2, the aprotic organic solvent is added to the reactant solution to crystallize a diastereomeric salt at −30 to 0° C.
